(19) United States
(12) Patent Application Publication
(10) Pub. No.: US 2021/0186376 A1
(43) Pub. Date: Jun. 24, 2021

(54) BIOFEEDBACK SYSTEM AND METHODS OF USING SAME

(71) Applicants: United States Government as Represented by the Department of Veterans Affairs, Washington, DC (US); Case Western Reserve University, Cleveland, OH (US); The MetroHealth System, Cleveland, OH (US)

(72) Inventors: BRIAN M. BECKER, Cincinnati, OH (US); MARK NANDOR, Cleveland Heights, OH (US); NATHANIEL MAKOWSKI, Cleveland, OH (US)

(21) Appl. No.: 17/127,000

(22) Filed: Dec. 18, 2020

Related U.S. Application Data
(60) Provisional application No. 62/949,788, filed on Dec. 18, 2019.

(51) Int. Cl. A61B 5/11; A61B 5/00

(52) U.S. Cl. CPC A61B 5/112; A61B 5/6828; A61B 5/6831; A61B 2505/09; A61B 5/486; A61B 5/7455; A61B 5/1116; A61B 5/1122

(57) ABSTRACT

A tactile biofeedback system includes is disclosed herein. The tactile biofeedback system can comprise an indicator and a sensor that is configured to be coupled to a user. A computing device can be in communication with the sensor and the indicator. The computing device can be configured to: receive a plurality of inertial measurements from the sensor, determine, based on the plurality of inertial measurements from the sensor, an occurrence of an improper movement, and cause the indicator to notify the user of the improper movement.

### CROSS-REFERENCE TO RELATED APPLICATIONS

This application claims priority to and the benefit of U.S. Provisional Patent Application No. 62/949,788, filed Dec. 18, 2019, the entirety of which is hereby incorporated by reference herein.

### FIELD

The disclosed invention relates to biofeedback systems and methods, and, in particular, to tactile biofeedback systems that facilitate gait rehabilitation.

### BACKGROUND

Conventionally, when promoting gait rehabilitation, physical therapists verbally instruct patients to bend their leg, and the patients need to think about what their leg is doing. While verbal cuing is effective in a hospital setting, patients do not always continue to follow these patterns in her home and community (beyond hospital based rehabilitation settings). If patients are not focused on proper gait (e.g., bending the knee), they can unconsciously develop bad habits.

Many patients with walking deficits from limb-loss, stroke, multiple sclerosis, or incomplete spinal cord injury use unhealthy compensatory strategies while they walk. For example, some patients use circumduction (swinging the leg out to the side) to generate toe clearance to prevent falls despite being capable of bending her hip and knee while it swings forward. In the short-term, this compensatory strategy effectively generates adequate toe clearance. Long term, this compensatory mechanism puts undue strain on other joints, causing joint pain and additional injury, which require additional treatment. Furthermore, the dysfunctional walking pattern can result in injurious falls.

There are some devices that assist with toe clearance, but each of these devices has deficiencies. An ankle foot orthosis is a plastic or metal brace that bends (dorsiflexes) the ankle to raise the toe and prevent it from dragging on the floor. Improved dorsiflexion can reduce the need for circumduction. Peroneal nerve stimulators use electrical stimulation to activate the muscles that generate dorsiflexion. The BIONESS L300Go incorporates a cuff that stimulates the hamstring muscles that can flex the leg. This approach directly activates the target muscles. Similarly, exoskeletons have been developed to directly assist with movements during walking. While these approaches are effective for patients who cannot generate the desired movement on her own, they are unlikely to be adopted by patients who can generate the desired movements without assistance, but need to be cued to remember to incorporate good habits.

### SUMMARY

Described herein, in various aspects, are biofeedback systems and methods.

In one aspect, a system can comprise an indicator and a sensor that is configured to be coupled to a user. A computing device can be in communication with the sensor and the indicator. The computing device can be configured to: receive a plurality of inertial measurements from the sensor, determine, based on the plurality of inertial measurements from the sensor, an occurrence of an improper movement, and cause the indicator to notify the user of the improper movement.

In exemplary aspects, the system can include a small, lightweight device worn on the thigh that measures thigh orientation while the leg swings forward and cues the patient with a sound or vibratory response when he or she circumducts the leg, swinging it out, instead of bending the leg appropriately while walking. The vibration or sound can cue the patient to flex his or her hip and knee instead of using compensatory strategies. In use, the device can reduce the prevalence of joint pain, falls and secondary injury, thereby improving patients' quality of life and saving money.

Also described are methods of using the disclosed systems.

### DESCRIPTION OF THE DRAWINGS

FIG. 1 is an image of an exemplary biofeedback system as disclosed herein.

FIG. 2 is a schematic diagram depicting the operation of the biofeedback system as disclosed herein.

FIG. 3 is a schematic diagram depicting an exemplary biofeedback system as disclosed herein.

FIG. 4 is a schematic diagram depicting an exemplary computing device in accordance with the present disclosure.

FIG. 5 is a schematic diagram showing a patient and illustrating abduction angle relative to a medial plane.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a”, “an”, and “the” include plural referents unless the context clearly dictates otherwise. For example, use of the term “a sensor” or “a measurement unit” can refer to one or more of such sensors or measurement units.

All technical and scientific terms used herein have the same meaning as commonly understood to one of ordinary skill in the art to which this invention belongs unless clearly indicated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about” or “substantially,” it is contemplated that values within up to 15%, up to 10%, up to 5%, or up to 1% (above or below) of the particularly stated value or characteristic can be included within the scope of those aspects.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

The following description supplies specific details in order to provide a thorough understanding. Nevertheless, the skilled artisan would understand that the apparatus and associated methods of using the apparatus can be implemented and used without employing these specific details. Indeed, the apparatus and associated methods can be placed into practice by modifying the illustrated apparatus and associated methods and can be used in conjunction with any other apparatus and techniques conventionally used in the industry.

Disclosed herein are devices and systems that can track movement of legs or other body parts during gait. For example, the device and systems can be configured to determine when patients (referred to herein also as “users”) make movements with their legs that add unnecessary wear and tear to the joints and/or make walking less efficient. In some optional aspects, a system 100 can comprise a sensor 102 such as, for example, a three-axis accelerometer (e.g., a three-degree-of-freedom accelerometer) or an inertial measurement unit (IMU) and a computing device 104 that is in communication with the sensor 102. The computing device 104 can receive a plurality of inertial measurements from the sensor 102 and determine, based on the inertial measurements, an occurrence of an improper movement (e.g., corresponding to a gait irregularity). It is contemplated that some IMUs (or other circuit board structures) have one or more sensors as well as integral computing architecture to process data from the one or more sensors. Accordingly, in some optional aspects, it is contemplated that the computing device 104, as further described herein can comprise computing architecture integral to the IMU (or other circuit board structure). Optionally, it is contemplated that the computing device 104 can further comprise one or more additional computing devices (e.g., a microprocessor) in communication with the IMU (or other circuit board structure). In further optional aspects, the computing device 104 can comprise or be embodied as a microcontroller.

The computing device 104 can further be in communication with an indicator 106. The indicator 106 can be, for example, but not limited to, a vibrational indicator, an audible indicator, or an electric stimulator. The indicator 106 can receive a signal from the computing device 104 in response to an improper movement occurring and, in response, provide an indication to the user. For example, the indicator 106 can be a vibration indicator comprising a motor 108, and the motor 108 can activate for a select period in response to receiving the signal from the computing device. It is contemplated that the motor can optionally produce a plurality of different vibrations to communicate different messages (e.g., improper movement, low battery, etc.). In further aspects, the audible indicator can comprise a speaker for producing an audible indication. The speaker can optionally be in communication with memory of the computing device, wherein the memory stores a sound file that can be played by the speaker. Accordingly, a processor of the computing device can cause the speaker to play the sound file. In some aspects, the sound can be mechanically generated via an actuator (optionally, without the need for a corresponding sound file). For example, the audible indicator can comprise a diaphragm and a transducer, and the computing device can provide an electrical signal that actuates the transducer. Optionally, for an audible indicator, different sounds can optionally correspond to different messages.

In some aspects, the system can further comprise a power source 110 (e.g., one or more batteries) that is configured to provide power to one or more of the sensor 102, the computing device 104, or the indicator 106.

In some aspects, the system can comprise an enclosure 112. Optionally, it is contemplated that the sensor 102, the computing device 104, and the indicator 106 can be housed within the enclosure 112. Accordingly, it is contemplated that the system 100 can be provided as a stand-alone device 120. In various further aspects, it is contemplated that one or more of the sensor 102, the computing device 104, or the indicator 106 can be provided as a separate component. For example, in various embodiments, the indicator 106 can be physically decoupled from the sensor 102. This can be advantageous, for example, for embodiments in which the sensor 102 is configured to couple to the shoe of the user. In such embodiments, the indicator 106 can optionally be spaced from the sensor 102 (e.g., coupled to the ankle). In various aspects, the computing device 104 can be in communication with the sensor 102 and the indicator 106 via wired or wireless communication. In some aspects, it is contemplated that the power source 110 can comprise a plurality of batteries that are positioned for providing power to the respective components.

In some optional aspects, components of a smartphone or other such device can embody at least one of the computing device or the indicator. For example, it is contemplated that the smartphone can wired or wirelessly receive inertial measurement data from the sensor 102, and the computing device of the smartphone can serve as the computing device 104 that determines the occurrence of an improper movement. In further aspects, it is contemplated that the smartphone can serve as the indicator 106. For example, the smartphone can provide the vibrational sensor or the speaker for providing indications to the user. Accordingly, in various optional aspects, the smartphone can be in communication with the sensor 102. In various further aspects, the smartphone can be in communication with a computing device 104 that is separate from the smartphone and that causes the indicator 106 (e.g., vibrational indicator) of the smartphone to activate.

In yet further aspects, it is contemplated that a smartphone can be adapted to serve as the computing device and indicator while also providing power for the system 100. In these aspects, it is contemplated that the smartphone can optionally couple to the sensor in a wired configuration (e.g., through a cable) to advantageously minimize delay in data transmission times.

In various further aspects, it is contemplated that the system 100 can be configured to detect and log each improper movement. The system can transmit (e.g., over certain intervals, intermittently, or when queried) the log of the improper movements to a remote device (e.g., smartphone, tablet, computer). In this way, the user can receive feedback about her walking pattern over time.

In further aspects, it is contemplated that a smartphone, tablet, computer, or other remote device can be configured to interface with the system 100 (optionally, wirelessly) to allow a user or clinician to set thresholds (e.g., acceptable and/or nonacceptable ranges of movement, numbers of improper movements within a defined time period, etc.) or change settings.

It is contemplated that various attachment features can be used to couple the sensor to a particular body part for measuring orientation of said body part during gait. For example, optionally, the system 100 can comprise a strap 114 that is configured to couple the sensor 102 to the user (such that the sensor is able to detect movement of body part of the user). For example, the strap 114 can be configured to extend around the leg or thigh of the user. The strap can comprise hook and loop, buckle, or other suitable fastener structures for releasably securing the fastener with a select circumference. The strap can optionally be elastic. In further aspects, the system 100 can comprise a clip that can be configured to clip to a shoe, belt, or pant waist. In still further aspects, other attachment features, such as adhesives, ties, clamps, harnesses, etc. can be used depending on the application. Optionally, the enclosure 112 can couple to the strap 114. For example, the enclosure can define at least one opening that is configured to receive a portion of the strap 114.

It is contemplated that the sensor can be coupled to the user at a known reference orientation. For example, the sensor can be positioned on a leg of the user in a particular fixed rotational orientation about the leg (e.g., centered on the front or back of the thigh) so that direction of movement of the leg can be determined. In this way, for example, abduction can be differentiated from front-to-back swinging.

Optionally, once the sensor is secured to the user, the sensor can be calibrated so that its initial orientation is known. For example, the user can stand in a predetermined orientation and then press a calibration button, and the computing device can store orientation values from the sensor as reference orientations. In further aspects, the sensor can be automatically calibrated. For example, the IMU or computing device can store, in memory, conventional calibration instructions that, when executed by at least one processor of the computing device, automatically determine orientation after movement during a calibration period.

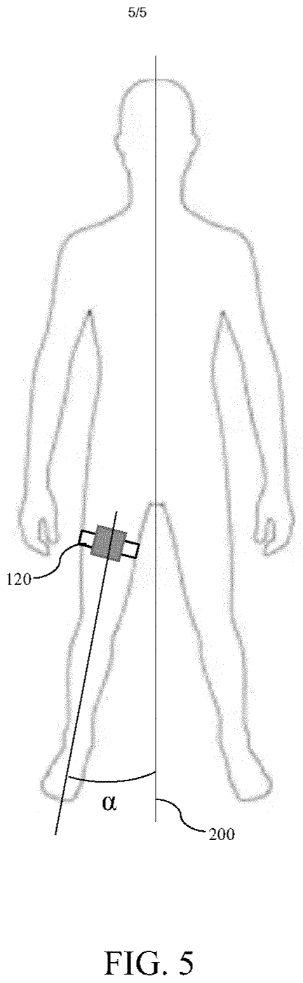

In various optional aspects, the sensor 102 (optionally, the device 120) can be worn around the thigh and can measure when a patient swings her leg out to the side to generate toe clearance instead of bending her knee. Such a movement can correspond to an abduction. As described herein and with reference to FIG. 5, abduction can refer to the thigh angle α relative to the medial plane 200. The system 100 can detect when the leg swings too far out to the side (excessive abduction) and, in response, can activate the indicator 106 (optionally, positioned on the back of the leg) to remind the patient to bend her knee more and not swing the leg out.

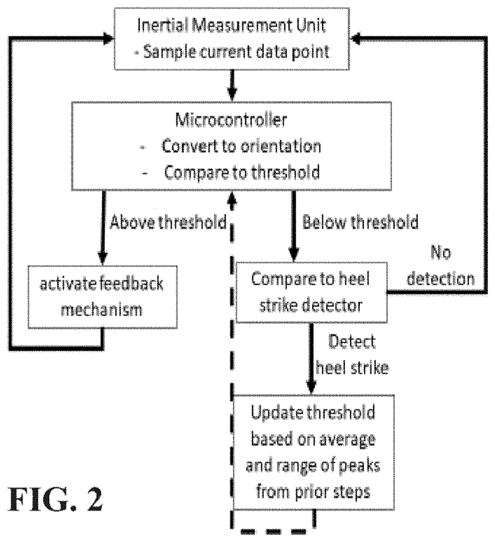

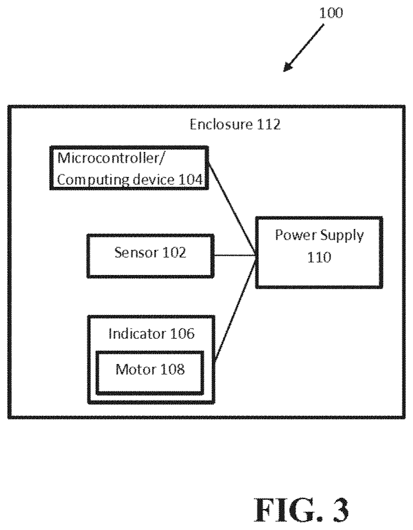

Referring to FIGS. 2 and 3, the system 100 can use the sensed data to determine a metric from the plurality of inertial measurements from the sensor 102, such as, for example, a measure of thigh orientation. For example, in some aspects, the sensor 102 (e.g., the IMU) can measure accelerations and velocities, and the computing device 104 can convert the accelerations and velocities to an orientation value, to thereby determine a measure of thigh orientation over time. For example, the sensor can comprise a three accelerometers that are all orthogonal to each other. Each accelerometer can change an electrical property (e.g., resistance) depending on the orientation of the accelerometer relative to a respective axis of measurement. Accordingly, the electrical property (e.g., resistance) across each accelerometer can be measured, and the measured resistance can correspond to an angle of orientation of each accelerometer relative to the respective axis. An algorithm can determine a common component of acceleration that corresponds to the acceleration due to gravity the direction thereof. The respective accelerations (i.e., acceleration relative to each axis) can be integrated over time in order to determine corresponding components of velocity. Additional features, such as, for example, gyroscopes, a compass, etc., can be incorporated to complement and improve accuracy of acceleration, velocity, and orientation data. It is contemplated that the sensor 102 can comprise a conventional, off-the-shelf IMU that is configured to measure and output orientation data. The computing device 104 can compare the metric (e.g., abduction angle) to a threshold. For example, the threshold can be a maximum abduction angle, and the abduction angle for the given step can be compared to the maximum abduction angle. Accordingly, in some aspects, the computing device can compare the thigh abduction component of orientation (or other metric) to the threshold to determine if the user is abducting more than she should be. If the metric (e.g., abduction angle) exceeds the threshold, the computing device can initiate an indication from the indicator. For example, the motor can be turned on for a set amount of time to cue the user to flex his or her knee instead of circumducting. In some optional aspects, the threshold for the abduction angle can be at least 5 degrees, at least 10 degrees, at least 15 degrees, at least 20 degrees, at least 25 degrees, or at least 30 degrees.

Optionally, the system 100 can determine, based on inertial measurement data from the sensor 102, a specific portion of a gait (e.g., between left toe lift and left heel strike), and the metric can be evaluated (e.g., compared to the threshold) only within the specific portion of the gait. This evaluation within only the specific portion of the gait can be valuable to detect, for example, steppage gait, as further described herein.

In some optional aspects, an algorithm (e.g., stored on memory in of the controller), can update the threshold (e.g., reduce the maximum leg abduction threshold) so that the patient can progressively learn to reduce how much she swings her leg out (the degree of abduction). Over time, the patient can learn to bend her knee appropriately and reduce bad habits. For example, the controller can automatically adjust the threshold based on at least one of a passage of a predetermined amount of time or a detection of an improvement. The improvement can be, for example, a change in frequency of steps in which the threshold is exceeded. In further aspects, the threshold can be updated dynamically based on a peak metric (e.g., peak abduction angle) measured during a prior step or a set of prior steps (e.g., over the course of several steps, ten minutes, an hour, a day, a week, a month, etc.) or based on a step in which the threshold is not exceeded. In some aspects, the system 100 can record a peak metric (e.g., abduction) for a particular step. For example, the IMU can detect heel strike, and the heel strike can be used to determine a transition between an end of a step and a beginning of a subsequent step. Based on the peak metric, and as depicted in FIG. 2, the threshold can be reduced (or, optionally, increased) as needed to adapt to the user's movements. For example, optionally, if the user does not surpass the threshold for one or more steps, the threshold can be reduced (e.g., by a set increment or as a function of a difference between the peak metric and the threshold). In further aspects, optionally, if the threshold is surpassed, the threshold can be increased (e.g., by a set increment or as a function of a difference between the peak metric and the threshold). A minimum threshold can be set so that, within a normal physiological range, the device will not provide an indication (e.g., activate the motor 108 FIG. 3). That is, the threshold can be limited not to drop below the minimum threshold. The minimum threshold can optionally correspond to a normal gait. For example, the minimum threshold can be five degrees for abduction. In further aspects, the threshold can be set by user input (e.g., by a physical therapist).

In use, the disclosed devices and systems can produce more efficient walking, a reduction in secondary injuries, and prevention of pain in other joints. More particularly, it is contemplated that through use of the system, patients can walk better and develop better walking habits. These improved walking habits can lead to reduced secondary care resulting from additional wear and tear on joints that results from moving in undesirable ways. Additional benefits include reduced joint pain, decreased usage of prescribed pain killers, reduced falls, reduced secondary injury, and increased mobility due to more efficient walking patterns.

It is further contemplated that the system can log data (e.g., number of occurrences of an improper movement, frequency of occurrences of improper movements, time of said occurrences, etc.). The data can be provided to a physician or therapist for analyzing progress. In exemplary aspects, the data can be logged in a remote computing device (e.g., a server). Optionally, in these aspects, the data can be selectively and/or remotely retrieved using Cloud-based data retrieval platforms.

Experimentally, it is contemplated that the performance of the disclosed devices and systems can be tested using the following protocols.

In laboratory testing First, a patient can walk without intentionally adapting gait pattern to measure hip abduction without actively attempting to adjust pattern. Then, the patient can walk while wearing the device and instructing her to bend her knee when she feel the vibration. Measure whether she flex her knee and reduces circumduction.

Home use testing Patients can take the system home and use it at home. Secondary injuries can be monitored, and walking can be measured at one month intervals to determine if there is an adjustment in knee flexion and circumduction both with and without the system.

### Use Cases

Particular use cases for the disclosed systems and methods are summarized below. Accordingly, although various embodiments are directed to use for minimizing abduction, embodiments can be adapted so that other cases, as further described herein, can be addressed to minimize improper gait.

Circumduction A patient swings her leg out to the side in order to generate toe clearance. The sensor can be worn on the thigh. In various other optional aspects, the sensor can be worn on other portions of a leg (e.g., the shank). A sensor can measure thigh abduction during swing, and values greater than a threshold be detected, and an indication (e.g., activation of the vibration motor worn on the back of the leg) can cue increased knee flexion and reduced hip abduction.

Vaulting A patient tilts her hips so that pelvic orientation is much higher than typical. The sensor can be worn on the belt, waist, or torso of the patient. The sensor can measure pelvic tilt during swing, and, if the computing device determines that one or more values exceed than a threshold, the computing device can cause an indicator to provide an indication (e.g., activation of the vibration motor worn on the pelvis near the belt) can cue increased knee flexion and decreased pelvic tilt. Optionally, a minimum threshold for vaulting can correspond to the pelvis rotating more than 5 degrees from horizontal. The threshold can optionally be at least 5 degrees, at least 10 degrees, at least 15 degrees, at least 20 degrees, at least 25 degrees, or at least 30 degrees from horizontal.

Steppage gait A patient exaggerates hip and knee flexion in swing because she does not generate adequate dorsiflexion. The sensor can be worn on the shoe or foot to measure foot orientation in swing. If the computing device detects that a value does not exceed a threshold within a certain portion of the gait cycle (as can be determined by the computing device), the computing device can cause the indicator to provide an indication (e.g., a vibration motor positioned on the front of the ankle can be activated) to cue proper dorsiflexion. Optionally, the minimum threshold can be correspond to the foot tilting more than 10 degrees below level or greater than 10 degrees below level.

Equinus gait A patient walks with an extended hip, knee, and ankle. The device can optionally be worn on the shoe or foot. The sensor can measure foot/ankle orientation in stance; the computing device can detect one or more values greater than a threshold and, in response cause the indicator to provide an indication (e.g., activate a vibrational motor on the ankle) to cue reduced tip toe gait. Optionally, a minimum threshold can be around zero degrees or slightly above zero degrees. The threshold can optionally be from zero to 60 degrees.

Trunk lean A patient tilts her trunk forward or to the side in order to shift center of gravity and maintain balance. The sensor can be worn on the trunk above the pelvis and can be configured to measure forward or lateral trunk lean. The computing device can detect one or more orientation values above a threshold and, in response, cause the indicator to provide an indication (e.g., activate a vibrational motor on the opposite side of the undesired tilt) to cue shifting body weight in the desired direction. The minimum threshold (corresponding to typical gait) can be about 5 or about 10 degrees in variation. For side tilt (lateral trunk lean), the threshold can optionally be in the range from about 5 degrees-30 degrees or about 10 degrees-30 degrees up to 30 degrees. For forward tilt (forward trunk lean), the threshold can range from about 5 degrees-60 degrees.

Recurvatum A patient hyperextends her knee for stability. The sensor can be worn on the shank (lower portion of the leg) to measure shank orientation during swing and stance. The computing device can detect one or more orientation values above a threshold and, in response, can cause the indicator to provide an indication (e.g., a vibrational motor worn on the back of the thigh can activate) to instruct a user to flex her knee more. Shank orientation for normal gait can correspond to a range from about 45 degrees to about −60 degrees. Recurvatum can correspond to the negative angle being smaller than normal gait.

It is contemplated that each of the thresholds herein can be tailored by clinicians/therapists or by algorithms that can be tailored, via internal programming or clinician/therapist input, to set maximum and/or minimum bounds for the thresholds.

As disclosed herein, the computing device can determine, based on feedback from the sensor 102, where the user is in the gait cycle. The gait cycle can include, for example, heelstrike, flat foot, midstance, pushoff, acceleration, mids-wing, and/or deceleration. In further aspects, the gait cycle can include initial contact, loading response, midstance, terminal stance, pre-swing, initial swing, mid-swing, and/or terminal swing. In various aspects, it is contemplated that certain abnormal gait patterns can incorporate measurements compared to a predetermined trajectory rather than a simple angle threshold. For example, the computing device can be programmed to include an ideal trajectory along the gait cycle. The ideal trajectory can comprise an orientation for at least one body part (e.g., hips, thigh, shank, or foot) for at least one portion of the gait cycle. The system 100 can determine the orientation of the at least one body part, and compare the at least one body part to the ideal trajectory. If the trajectory deviates from the ideal trajectory by a threshold, the system can provide an indication. Such feedback can be advantageously used for gait irregularities such as steppage gait and recurvatum, in which certain orientations are proper during portions of the gait cycle and improper during other portions of the gait cycle.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

### Computing Device

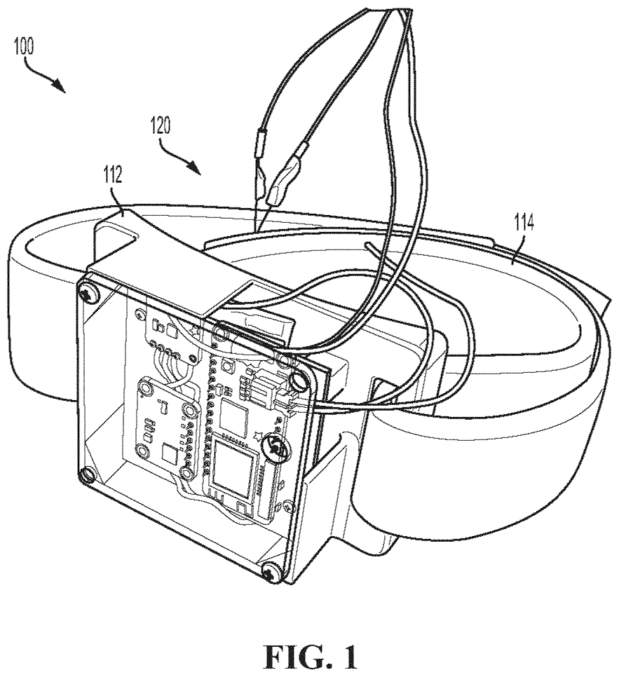

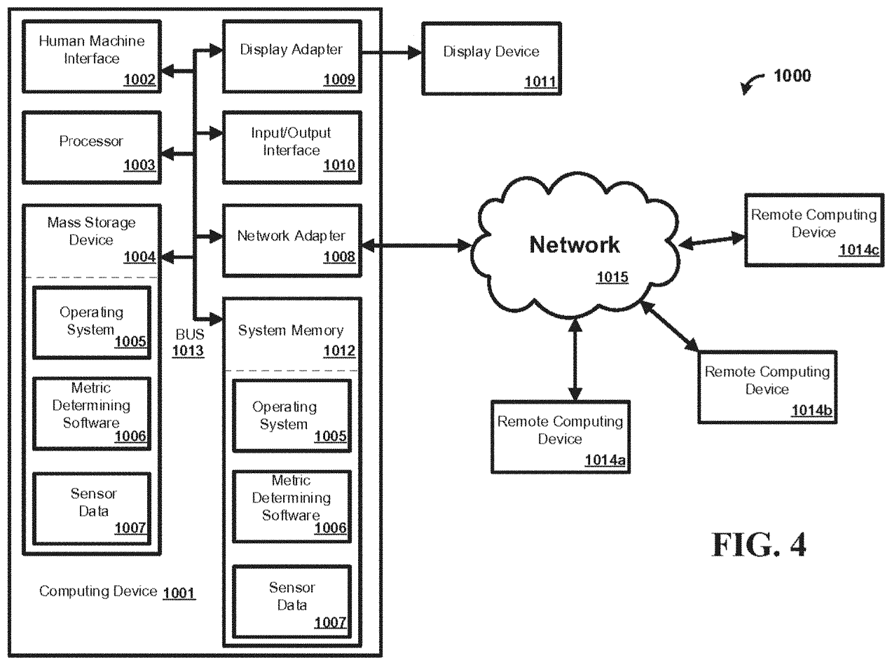

FIG. 4 shows an operating environment 1000 including an exemplary configuration of a computing device 1001 that, in some embodiments, can be the computing device 104. In further aspects, the computing device 104 (e.g., the microcontroller of the device 120, FIG. 1) can be a portion of the computing device 1001. That is, in some optional aspects, various computing devices can cooperatively be used with the system 100.

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as sensor data 1007 and/or program modules such as operating system 1005 and metric determining software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and metric determining software 1006 may be stored on the mass storage device 1004. One or more of the operating system 1005 and metric determining software 1006 (or some combination thereof) may comprise program modules and the metric determining software 1006. Sensor data 1007 may also be stored on the mass storage device 1004. Sensor data 1007 may be stored in any of one or more databases known in the art. The databases may be centralized or distributed across multiple locations within the network 1015.

A user may enter commands and information into the computing device 1001 using an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a,b,c may be made using a network 1015, such as a local area network (LAN) and/or a general wide area network (WAN). Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a,b,c can optionally have some or all of the components disclosed as being part of computing device 1001.

Application programs and other executable program components such as the operating system 1005 are shown herein as discrete blocks, although it is recognized that such programs and components may reside at various times in different storage components of the computing device 1001, and are executed by the one or more processors 1003 of the computing device 1001. An implementation of data processing software 1006 may be stored on or sent across some form of computer readable media. Any of the disclosed methods may be performed by processor-executable instructions embodied on computer readable media.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A system for a user, the system comprising: a sensor that is configured to be coupled to the user; an indicator; a computing device in communication with the sensor and the indicator, wherein the computing device is configured to: receive a plurality of inertial measurements from the sensor; determine, based on the plurality of inertial measurements from the sensor, an occurrence of an improper movement; and cause the indicator to notify the user of the improper movement.

Aspect 2: The system of aspect 1, wherein the sensor comprises an inertial measurement unit (IMU).

Aspect 3: The system of aspect 1 or aspect 2, wherein the sensor comprises a three-axis accelerometer.

Aspect 4: The system of any one of the preceding aspects, wherein the indicator comprises a motor that is configured to cause a vibration.

Aspect 5: The system of any one of the preceding aspects, further comprising a strap that is configured to secure the sensor to a leg of the user.

Aspect 6: The system of aspect 5, further comprising an enclosure coupled to the strap, wherein the enclosure houses the sensor, the indicator, and the computing device.

Aspect 7: The system of any one of the preceding aspects, wherein the improper movement corresponds to an outward thigh swing.

Aspect 8: The system of aspect 7, wherein the computing device is configured to determine the occurrence of the improper movement based on a metric surpassing a threshold.

Aspect 9: The system of aspect 8, wherein the metric is an abduction angle.

Aspect 10: The system of aspect 8, wherein the metric is a foot orientation angle.

Aspect 11: The system of aspect 8, wherein the metric is a trunk lean angle.

Aspect 12: The system of aspect 8, wherein the metric is a shank orientation angle.

Aspect 13: The system of aspect 8, wherein the metric is compared to the threshold during a select portion of a gait.

Aspect 14: The system of aspect 8, wherein the threshold is adjustable.

Aspect 15: The system of aspect 14, wherein the controller is configured to automatically adjust the threshold based on at least one of: a passage of a predetermined amount of time; or a detection of an improvement.

Aspect 16: The system of aspect 15, wherein the improvement comprises one of: a change in frequency of steps in which the threshold is exceeded; or a step in which the metric does not exceed the threshold.

Aspect 17: The system of aspect 16, wherein the computing device is configured to determine an occurrence of the step in which the metric does not exceed the threshold and, reduce the threshold in response to the occurrence.

Aspect 18: The system of any one of aspects 14-17, wherein the threshold is adjustable based on user input.

Aspect 19: A method using the system as in any one of aspects 1-18, the method comprising: coupling the sensor to a leg of the user.

Aspect 20: The method of aspect 20, wherein user has a thigh, wherein coupling the sensor to the leg comprises coupling the sensor to the thigh of the user.

Although the foregoing invention has been described in some detail by way of illustration and example for purposes of clarity of understanding, certain changes and modifications may be practiced within the scope of the appended claims.

What is claimed is:

1. A system for a user, the system comprising:
a sensor that is configured to be coupled to the user;
an indicator;
a computing device in communication with the sensor and the indicator, wherein the computing device is configured to:
receive a plurality of inertial measurements from the sensor;
determine, based on the plurality of inertial measurements from the sensor, an occurrence of an improper movement; and
cause the indicator to notify the user of the improper movement.

2. The system of claim 1, wherein the sensor comprises an inertial measurement unit (IMU).

3. The system of claim 1, wherein the sensor comprises a three-axis accelerometer.

4. The system of claim 1, wherein the indicator comprises a motor that is configured to cause a vibration.

5. The system of claim 1, further comprising a strap that is configured to secure the sensor to a leg of the user.

6. The system of claim 5, further comprising an enclosure coupled to the strap, wherein the enclosure houses the sensor, the indicator, and the computing device.

7. The system of claim 1, wherein the improper movement corresponds to an outward thigh swing.

8. The system of claim 7, wherein the computing device is configured to determine the occurrence of the improper movement based on a metric surpassing a threshold.

9. The system of claim 8, wherein the metric is an abduction angle.

10. The system of claim 8, wherein the metric is a foot orientation angle.

11. The system of claim 8, wherein the metric is a trunk lean angle.

12. The system of claim 8, wherein the metric is a shank orientation angle.

13. The system of claim 8, wherein the metric is compared to the threshold during a select portion of a gait.

14. The system of claim 8, wherein the threshold is adjustable.

15. The system of claim 14, wherein the controller is configured to automatically adjust the threshold based on at least one of:
a passage of a predetermined amount of time; or
a detection of an improvement.

16. The system of claim 15, wherein the improvement comprises one of:
a change in frequency of steps in which the threshold is exceeded; or
a step in which the metric does not exceed the threshold.

17. The system of claim 16, wherein the computing device is configured to determine an occurrence of the step in which the metric does not exceed the threshold and, reduce the threshold in response to the occurrence.

18. The system of claim 14, wherein the threshold is adjustable based on user input.

19. A method comprising: coupling a sensor of a system to a leg of a user, wherein the system comprises:
the sensor;
an indicator;
a computing device in communication with the sensor and the indicator, wherein the computing device is configured to:
receive a plurality of inertial measurements from the sensor;
determine, based on the plurality of inertial measurements from the sensor, an occurrence of an improper movement; and
cause the indicator to notify the user of the improper movement

20. The method of claim 19, wherein user has a thigh, wherein coupling the sensor to the leg comprises coupling the sensor to the thigh of the user.
